(19) United States
(12) Patent Application Publication
(10) Pub. No.: US 2020/0306252 A1
(43) Pub. Date: Oct. 1, 2020

(54) COMPOSITION FOR PREVENTING AND/OR TREATING DEMENTIA

(71) Applicant: Eric Yueh-Lang SHEU, Ji'an Township (TW)

(72) Inventor: Eric Yueh-Lang SHEU, Ji'an Township (TW)

(21) Appl. No.: 16/510,639

(22) Filed: Jul. 12, 2019

(30) Foreign Application Priority Data
Apr. 1, 2019 (TW) 108111508

(51) Int. Cl. A61K 31/522; A61K 31/122; A61K 31/353; A61K 31/366; A61K 31/19; A61K 31/047; A61K 31/015; A61P 25/28

(52) U.S. Cl. CPC A61K 31/522; A61K 31/122; A61K 31/353; A61P 25/28; A61K 31/19; A61K 31/047; A61K 31/015; A61K 31/366

(57) ABSTRACT

A composition, comprising a first component and a second component, wherein the first component is at least one of xanthine and a derivative of xanthine, and the second component is at least one of diarylheptanoid, a derivative of diarylheptanoid, catechin, an ester of catechin, flavonoid, and isoflavonoid, and wherein the first component is optionally combined with the second component as an adduct. The composition could be used to treat and/or prevent dementia.

### CLAIM FOR PRIORITY

This application claims the benefit of Taiwan Patent Application No. 108111508 filed on Apr. 1, 2019, the subject matter of the application is incorporated herein in its entirety by reference.

### FIELD OF THE INVENTION

The present invention relates to a composition, comprising a first component and a second component, wherein the first component is at least one of xanthine and a derivative of xanthine, and the second component is at least one of diarylheptanoid, a derivative of diarylheptanoid, catechin, an ester of catechin, flavonoid, and isoflavonoid, and wherein the first component is optionally combined with the second component as an adduct. The present invention also relates to the uses of the composition in treating and/or preventing dementia, including using the composition in the manufacture of a medicament for treating and/or preventing dementia, and using the composition in a method for treating and/or preventing dementia.

### BACKGROUND OF THE INVENTION

Dementia is a syndrome comprising various brain function degenerations and disabilities. Patients with dementia will show degeneration in language ability, spatial perception, arithmetic ability, judgment, abstract thinking and attention, and may show disruptive behaviors, personality changes, delusions and hallucination. The aforesaid symptoms will become severe along with the progression of dementia, and gradually affect the daily life and interpersonal relationship of patients. Patients with late-stage dementia may not be able to live independently, and may need a full time caretaker. Therefore, dementia does not only involve the patient himself or herself but other caregiving parties. The number of patients with dementia continues to increase with the worldwide aging population, and thus, the high cost of care cannot be ignored.

Dementia can be classified into the following four types: degenerative dementia, vascular dementia, mixed (both degenerative and vascular) dementia and reversible dementia, wherein only the reversible dementia belongs to temporary dementia, which is caused by other diseases and can be cured when the causes of diseases are removed. Degenerative dementia includes Alzheimer's disease, frontotemporal lobe degeneration, dementia with Lewy Bodies, Parkinson's disease, etc., wherein the dementia with Lewy Bodies and Parkinson's disease have similar pathogenesis (e.g., an abnormal aggregation of Lewy Bodies), and similar clinical characteristics. Other pathogenesis related to dementia includes β-amyloid aggregation, τ protein phosphorylation, blood and brain dysfunction, viral infections, etc. Since the specific mechanisms of dementia are still poorly understood, it is hard to design an effective drug for treating dementia.

Currently, the drugs being developed for dementia include NMDA receptor antagonists (e.g., memantine), and acetylcholinesterase inhibitors (e.g., donepezil, rivastigmine, galantamine). However, each of the aforesaid drugs can only regulate a single neurotransmitter while dementia likely involves multiple neurotransmitter pathways. These can be noted from such as “Alisky J M., Neurotransmitter depletion may be a cause of dementia pathology rather than an effect. Med Hypotheses. 2006, 67(3):556-60, which is entirely incorporated hereinto by reference. Although the symptoms of dementia may be temporarily alleviated by the market-available drugs, they cannot prevent the progression of neurodegeneration nor be effective at improving symptoms as degeneration progresses to later stages.

In addition to the current market drugs for dementia, multiple attempts have been made to develop drugs that target beta-secretase (BACE), an enzyme responsible for producing beta-amyloid. However, these drugs have not seen success in clinical trials. Pfizer's (BACE) inhibitor bapineuzemab failed clinical trial Phase III in 2012 due to “no clinical benefit” to the primary measures as reported by Pollack, A.: “Alzheimer's Drug Fails its First Clinical trial”; and lanabecestat by AstraZeneca and Eli Lilly failed clinical trial Phase III due to “failure to meet primary endpoints” as reported by Drug Development Technology: “Another BACE Inhibitor Fails Phase III Trials.” On top of not being efficacious, BACE inhibitors pose several safety issues: adverse effects on other organ systems, such as the liver, have been reported by Taylor, P: “Janssen drops the BACE as Alzheimer's candidate joins fail list.” These findings on the failures of BACE-inhibiting drugs are summarized in a 2018 report (Panza, F et al. “Are Antibodies Directed Against Amyloid-β (A(β) Oligomers the Last Call for the AP Hypothesis of Alzheimer's Disease?” Immunotherapy (2019) 11(1), 3-6, 7 Dec. 2018), which is entirely incorporated hereinto by reference.

Therefore, in addition to the above mentioned chemically synthesized drugs or market-available drugs, an approach using botanical extracts is highly desirable in the art. Some compounds extracted from plants have been discovered as having a potential to be developed into the drug for treating and/or preventing dementia. In cellular experiments, curcumin is effective in protecting neural cells and reducing β-amyloid aggregation, and thus, is considered as a potential compound. However, the results of the clinical experiments show that curcumin alone cannot effectively alleviate the symptoms of patients with dementia (these can be noted from Goozee et al.: “Examining the potential clinical value of curcumin in the prevention and diagnosis of Alzheimer's disease. Br J Nutr. 115, 449-465,” which is entirely incorporated hereinto by reference). Furthermore, researchers found that curcumin cannot penetrate the blood-brain barrier (BBB) effectively and thus, has poor bioavailability.

Inventors of the present invention discovered that xanthine and its derivatives are capable of carrying other components across the BBB. Therefore, the present invention provides a composition comprising a first component and a second component, wherein the first component is at least one of xanthine and a derivative of xanthine, and the second component is at least one of diarylheptanoid, a derivative of diarylheptanoid, catechin, an ester of catechin, flavonoid, and isoflavonoid, and wherein the first component is optionally combined with the second component as an adduct. By using the aforesaid first component, which can carry the second component across the BBB, in combination with the second component, the present invention could provide an effect of preventing and/or treating dementia. On the other hand, the present invention administers various compounds at one administration to target various mechanisms of dementia at the same time, thereby, enhancing the efficiency of preventing and/or treating dementia.

### SUMMARY OF THE INVENTION

One objective of the present invention is to provide a composition, comprising a first component and a second component, wherein the first component is at least one of xanthine and a derivative of xanthine, and the second component is at least one of diarylheptanoid, a derivative of diarylheptanoid, catechin, an ester of catechin, flavonoid, and isoflavonoid, the first component is optionally combined with the second component as an adduct.

Generally, based on the total weight of the first component and the second component, the amount of the first component ranges from 0.025 wt % to 25 wt %. Preferably, based on the total weight of the first component and the second component, the amount of the first component ranges from 0.05 wt % to 10 wt %. More preferably, based on the total weight of the first component and the second component, the amount of the first component ranges from 0.1 wt % to 5 wt %. The amount of the first component refers to the total amount of the first component being combined with the second component as an adduct and the first component in free form (i.e., not combined with the second component). The total amount of the first component and second component refers to the total amount of the adduct of the first component and the second component, the first component in free form, and the second component in free form. Specifically, if the entire first component is combined with the second component as an adduct, the amount of the first component refers to the amount of the moiety in the adduct that is from the first component. On the other hand, if only a part of the first component is combined with the second component as an adduct, the amount of the first component refers to the total amount of the first component in free form and the moiety in the adduct that is from the first component.

Preferably, the first component of the composition in accordance with the present invention is at least one of caffeine, aminophylline, 3-isobutyl-1-methylxanthine (IBMX), paraxanthine, pentoxifylline, theobromine, and theophylline.

Among the materials suitable for the second component in the composition in accordance with the present invention, the diarylheptanoid is preferably curcumin; the derivative of diarylheptanoid is preferably at least one of curcuminoid, demethoxycurcumin and bisdemethoxycurcumin; the ester of catechin is preferably epigallocatechin gallate (EGCG); the flavonoid is preferably at least one of apigenin, anthocyanin, anthraquinone, quercetin and luteolin; and the isoflavonoid is preferably genistein, daidzein and glycitein.

Preferably, the composition in accordance with the present invention further comprises a third component, wherein the third component is at least one of a carotenoid, zeaxanthin, lycopene, carotene, carotenoid crocetin, carotenoid acid, and ω-3 fatty acid.

Another objective of the present invention is to provide the use of the above composition in the manufacture of a medicament, wherein the medicament is used for treating and/or preventing dementia. Preferably, the dementia is at least one of Alzheimer's disease, and Parkinson's disease.

Yet another objective of the present invention is to provide a method for treating and/or preventing dementia, comprising administering an effective amount of the above composition to a subject in need thereof. Preferably, the dementia is at least one of Alzheimer's disease and Parkinson's disease.

The detailed technology and preferred embodiments implemented for the present invention will be described in the following paragraphs for persons skilled in the art to well appreciate the features of the claimed invention.

### BRIEF DESCRIPTION OF THE DRAWINGS

The patent application contains at least one drawing executed in color. Copies of this patent with color drawing(s) will be provided by the Patent and Trademark Office upon request and payment of the necessary fee.

FIGS. 1A to 1F show the influence of the composition of the present invention on the Aβ₁₋₄₂ protein aggregation in the hippocampal CA1 region, wherein FIG. 1A shows the results of the untreated SD rats (i.e., “Control” group); FIG. 1B shows the results of the SD rats subjected to a perfusion with FAB (ferrous amyloid buthionine) solution (i.e., “FAB” group); FIG. 1C shows the results of the SD rats subjected to a perfusion with FAB solution and an intraperitoneal injection with a composition comprising curcumin, EGCG and apigenin (i.e., “Formulation 1” group); FIG. 1D shows the results of the SD rats subjected to a perfusion with FAB solution and an intraperitoneal injection with a composition comprising curcumin, EGCG, anthocyanin and caffeine (i.e., “Formulation 2” group); FIG. 1E shows the results of the SD rats subjected to a perfusion with FAB solution and an intraperitoneal injection with a composition comprising curcumin, EGCG, anthraquinone and caffeine (i.e., “Formulation 3” group); and FIG. 1F is a bar graph showing the quantified results of FIGS. 1A to 1E (proportional scale: 50 μm; * represents the result is significantly different from that of the “Control” group, p<0.05; # represents the result is significantly different from that of the “FAB” group, p<0.05).

FIGS. 2A to 2F show the influence of the composition of the present invention on the microglia density in the hippocampal CA1 region, wherein FIGS. 2A to 2E are photographs respectively showing the results of the above “Control” group, “FAB” group, “Formulation 1” group, “Formulation 2” group and “Formulation 3” group, and FIG. 2F is a bar graph showing the quantified results of FIGS. 2A to 2E (proportional scale: 100 μm; * represents the result is significantly different from that of the “Control” group, p<0.05).

FIGS. 3A to 3F show the influence of the composition of the present invention on the survival rate of pyramidal neurons in the hippocampal CA1, wherein FIGS. 3A to 3E are photographs respectively showing the results of the above “Control” group, “FAB” group, “Formulation 1” group, “Formulation 2” group and “Formulation 3” group, and FIG. 3F is a bar graph showing the quantified results of FIGS. 3A to 3E (proportional scale: 100μm; * represents the result is significantly different from that of the “Control” group, p<0.05; # represents the result is significantly different from that of the FAB group, p<0.05).

FIGS. 4A to 4F show the influence of the composition of the present invention on the dendritic spine density on the distal apical dendrite of pyramidal neurons in the hippocampal CA1 region, wherein FIGS. 4A to 4E are photographs respectively showing the results of the above “Control” group, “FAB” group, “Formulation 1” group, “Formulation 2” group and “Formulation 3” group, and FIG. 4F is a bar graph showing the quantified results of FIGS. 4A to 4E (proportional scale: 10 μm; * represents the result is significantly different from that of the “Control” group, p<0.05; # represents the result is significantly different from that of the “FAB” group, p<0.05).

FIGS. 5A to 5E show the influence of the composition of the present invention on the number of cholinergic neurons in the medial septal nucleus, wherein FIGS. 5A to 5D are photographs respectively showing the results of the above “Control” group, “FAB” group, “Formulation 1” group, “Formulation 2” group and “Formulation 3” group, and FIG. 5E is a bar graph showing the quantified results of FIGS. 5A to 5D (proportional scale: 200 μm; * represents the result is significantly different from that of the “Control” group, p<0.05; # represents the result is significantly different from that of the “FAB” group, p<0.05).

FIG. 6 shows the UV-VIS spectrums of quercetin-caffeine adduct of the present invention and quercetin, wherein the blue line is the spectrum of quercetin-caffeine adduct and the red line is the spectrum of quercetin.

FIG. 7 shows the UV-VIS spectrums of apigenin-caffeine adduct of the present invention and apigenin, wherein the blue line is the spectrum of apigenin-caffeine adduct and the red line is the spectrum of apigenin.

### DESCRIPTION OF THE PREFERRED EMBODIMENT

The following paragraphs will describe some of the embodiments of the present invention in detail. However, without departing from the spirit of the present invention, the present invention may be embodied in various embodiments and should not be limited to the embodiments described in the specification.

Unless otherwise indicated herein, the expression “a,” “an,” “the,” or the like recited in this specification (especially in the claims) are intended to include both the singular and plural forms. The term “prevent” or “preventing” recited in this specification refers to inhibiting or avoiding a particular condition of illness from breaking out, maintaining good health in a sensitive subject, or establishing the ability of a sensitive subject to tolerate diseases. The term “treat”, or “treating” recited in this specification should not be construed as treating a subject until the subject is completely recovered, but should include maintaining the progression or symptoms of the diseases in a substantially static state, increasing the recovery rate of a subject, alleviating the severity of a particular condition of illness, or enhancing the life quality of patients. The term “subject” recited in this specification refers to human and non-human mammalians.

The numerical ranges (e.g., 5 to 100) used in this specification should be construed as including all of the rational numbers in the ranges and ranges consisting of any rational numbers in the ranges. Therefore, the numerical ranges used in this specification should include all the possible combinations of numerical values between the lowest value and the highest value listed therein.

As described above, inventors of the present invention discovered that xanthine and its derivatives are capable of carrying other components across the BBB, and thus, using xanthine and its derivatives in combination with the potential compounds of treating or preventing dementia could improve the bioavailability of such potential compounds.

Therefore, the present invention relates to a composition, comprising a first component and a second component, wherein the first component is at least one of xanthine and a derivative of xanthine, and the second component is at least one of diarylheptanoid, a derivative of diarylheptanoid, catechin, an ester of catechin, flavonoid, and isoflavonoid, and the first component is optionally combined with the second component as an adduct. The present invention also relates to the uses of the composition in treating and/or preventing dementia, including using the composition in the manufacture of a medicament for treating and/or preventing dementia, and using the composition in a method for treating and/or preventing dementia.

The derivatives of xanthine suitable for the first component of the composition in accordance with the present invention could be methylxanthine, including caffeine, aminophylline, 3-isobutyl-1-methylxanthine (IBMX), paraxanthine, pentoxifylline, theobromine, and theophylline.

Diarylheptanoid suitable for the second component of the composition in accordance with the present invention could be a linear diarylheptanoid or a circular diarylheptanoid, wherein the examples of linear diarylheptanoid include, but not limited to, curcumin. Examples of the derivatives of diarylheptanoid include, but not limited to, curcuminoid, demethoxycurcumin, bisdemethoxycurcumin, and other derivatives of curcumin.

Catechin and the esters of catechin suitable for the second component of the composition in accordance with the present invention could be extracted from a plant. For example, the catechin and the esters of catechin could be extracted from teas (green tea or black tea), apple peels, plums, onions, hazelnuts, carob beans, etc., but is not limited thereby. Preferably, the ester of catechin suitable for the second component of the composition, is EGCG.

The flavonoid suitable for the second component of the composition in accordance with the present invention could be a secondary metabolite of a plant or a fungus (i.e., a bioflavonoid) and has two benzene rings and a heterocyclic ring in its structure. For example, the flavonoid suitable for the second component could be selected from the group consisting of anthoxanthin, flavonol, flavone, flavanone, astragalosides I to IV, flavan, flavanol, anthocyanin, anthocyanidin, neoflavonoid, their derivatives, their esters, and combinations thereof. In some embodiments of the present invention, the second component of the composition is at least one of apigenin, anthocyanin, anthraquinone, quercetin, and luteolin. In addition, examples of the isoflavonoid suitable for the second component of the composition include, but are not limited to, genistein, daidzein, and glycitein.

In one embodiment of the present invention, caffeine was used as the first component, and EGCG, anthocyanin and curcumin were used as the second component. In another embodiment of the present invention, caffeine was used as the first component, and EGCG, anthraquinone and curcumin were used as the second component.

There is no particular limitation of the ratio of amount of the first component to that of the second component in the composition in accordance with the present invention. In general, based on the total weight of the first component and the second component, the amount of the first component ranges from 0.025 wt % to 25 wt %, preferably from 0.05 wt % to 10 wt %, and more preferably from 0.1 wt % to 5 wt %. In one embodiment of the present invention, based on the total weight of the first component and the second component, the amount of the first component is 0.5 wt %.

The composition in accordance with the present invention could optionally further comprise a third component or be used in combination with a drug or a food containing the third component to further enhance the effects of the composition or to increase the application flexibility and application adaptability of the preparation thus provided. The third component should not adversely affect the effects of the first component and second component. Examples of the third component include, but are not limited to, carotenoid, zeaxanthin, lycopene, carotene, carotenoid crocetin, carotenoid acid, and ω-3 fatty acid. The third component could be extracted from a plant, and examples of the plants include, but are not limited to, crocus sativus and its analogs (e.g., saffron), legumes, celery, blueberry, coffee beans, dark chocolate, pumpkin seeds, broccoli, various nuts, orange or its families.

Preferably, in the composition of the present invention, the first component is combined with the second component as an adduct, to ensure the second component could across the BBB efficiently, thereby, fully elaborating the effects of treating and/or preventing dementia. The first component is combined with the second component by a hydrogen bond, so that the adduct would contain all of the atoms of the first component and second component. Examples of the first component include xanthine and the derivatives of xanthine. Examples of the second component include diarylheptanoid, the derivatives of diarylheptanoid, catechin, the esters of catechin, flavonoid, and isoflavonoid. Both the first component and second component should have a hydrogen bond-forming group (e.g., OH and N).

For example, in the composition of the present invention, when the first component and the second component are present in the form of an adduct, the first component could be caffeine, and the second component could be at least one of rosemaric acid, kaempferol, quercetin, delphinidin chloride and apigenin, but are not limited thereby. In one embodiment of the present invention, caffeine, as the first component, and rosemaric acid, as the second component, are combined as an adduct. In another embodiment of the present invention, caffeine, as the first component, and kaempferol, as the second component, are combined as an adduct. In still another embodiment of the present invention, caffeine, as the first component, and quercetin, as the second component, are combined as an adduct. In yet another embodiment of the present invention, caffeine, as the first component, and delphinidin chloride, as the second component, are combined as an adduct. In yet another embodiment of the present invention, caffeine, as the first component, and apigenin, as the second component, are combined as an adduct.

In some embodiments of the present invention, the adduct in the composition of the present invention could be provided by dissolving the first component and second component in a solvent to provide a reaction mixture and then removing the solvent therefrom. Examples of the solvent include, but are not limited to, toluene, carbon disulfide, ethyl acetate, methanol, ethanol and water. To promote the formation of adduct, before removing the solvent from the mixture, an operation such as heating and reflux could be optionally conducted.

The composition in accordance with the present invention could be used for treating and/or preventing dementia, wherein the dementia includes degenerative dementia, vascular dementia, mixed dementia and reversible dementia. Examples of degenerative dementia include, but are not limited to, Alzheimer' s disease, Parkinson's disease, frontotemporal lobe degeneration and dementia with Lewy Bodies.

The composition in accordance with the present invention is a pharmaceutical composition or a food composition. Depending on the desired purpose, the pharmaceutical composition could be provided in any suitable form without any particular limitations. For example, the composition in accordance with the present invention could be administered to a subject in need by an oral, rectal, transdermal, ophthalmic, inhalation, subcutaneous, intramuscular or intravenous route, but is not limited thereby.

The pharmaceutical composition in accordance with the present invention could be administered to a subject in need systemically or topically, and could be delivered by various drug delivery systems (DDSs), such as an oral drug delivery system, rectal drug delivery system, transdermal drug delivery system, ophthalmic drug delivery system, inhalation drug delivery system, etc. The composition in accordance with the present invention could also be administered by infusion, injection, implantation, or surgery. For example, to enhance bioavailability, control drug release speed, target the lesion precisely and reduce side effects, the pharmaceutical composition could be delivered by a liposome, a microcapsule, nanoparticles, or microneedles, but is not limited thereby.

Depending on the form and purpose(s), suitable carriers could be chosen and used to provide the pharmaceutical composition. Examples of the carriers include, but are not limited to, excipients, diluents, auxiliaries, stabilizers, absorbent retarders, disintegrating agents, hydrotropic agents, emulsifiers, antioxidants, adhesives, binders, tackifiers, dispersants, suspending agents, lubricants, and hygroscopic agents.

As a form suitable for oral administration, the pharmaceutical composition in accordance with the present invention could comprise any pharmaceutically acceptable carrier that will not adversely affect the desired effects of the active ingredients (i.e., the first component and the second component). Examples of the carrier include, but are not limited to, water, saline, dextrose, glycerol, ethanol or its analogs, cellulose, starch, sugar bentonite, oil (e.g., olive oil, castor oil, cottonseed oil, peanut oil, corn oil, and germ oil), polyethylene glycol, and combinations thereof. The pharmaceutical composition could be provided by any suitable method in any suitable form for oral administration, such as the form of a tablet (e.g., sugar-coated tablet), a pill, a capsule, a powder, granules, a pulvis, a fluid extract, a solution, a syrup, a suspension, a tincture, but is not limited thereby.

As for the form suitable for rectal administration, the pharmaceutical composition could also comprise any pharmaceutically acceptable carrier that will not adversely affect the desired effects of the active ingredients (i.e., the first component and the second component). Examples of the carrier include, but are not limited to, lactose, corn starch, talc, cellulose, sucrose powder, magnesium stearate, mannitol, light anhydride silicic acid, magnesium carbonate, calcium carbonate, L-cysteine, starch, sucrose, gelatin, arabic gum powder, methylcellulose, carboxymethyl cellulose, sodium carboxymethyl cellulose, hydroxypropyl cellulose, hydroxypropyl methylcellulose, polyvinylpyrrolidone, amylopectin, dextrin, and combinations thereof. The pharmaceutical composition could be provided by any suitable method in any suitable form for rectal administration, such as in the form of a capsule, a tablet, a solution, a suppository, but is not limited thereby.

As for the form suitable for transdermal administration, the pharmaceutical composition could also comprise any pharmaceutically acceptable carrier that will not adversely affect the desired effects of the active ingredients (i.e., the first component and the second component). Examples of the carrier include, but are not limited to, water, mineral oil, propanediol, polyethylene oxide, liquid petrolatum, sorbitan monostearate, and polysorbate 60. The pharmaceutical composition could be provided by any suitable method in any suitable form for transdermal administration, such as an emulsion (e.g., a massage emulsion), a cream (e.g., a massage cream), an oil (e.g., a massage oil), a gel (e.g., a hydrogel), a paste (e.g., a dispersing paste, an ointment), a lotion, a spray, or a patch (e.g., a microneedle patch), but is not limited thereby.

As for the form suitable for ophthalmic administration, the pharmaceutical composition could also comprise any pharmaceutically acceptable carrier that will not adversely affect the desired effects of the active ingredients (i.e., the first component and the second component). Examples of the carrier include, but are not limited to, surfactant, viscosity modifier, osmotic pressure modifier, buffer, and water. The pharmaceutical composition could be provided by any suitable method in any suitable form for ophthalmic administration, such as an emulsion, an oil, a gel, an ointment, a solution, a suspension, but is not limited thereby.

As for the form suitable for inhalation administration, the pharmaceutical composition could also comprise any pharmaceutically acceptable carrier that will not adversely affect the desired effects of the active ingredients (i.e., the first component and the second component). Examples of the carrier include, but are not limited to, sugar, alcohol, amino acid, phospholipid, surfactant, cyclodextrin, high molecular substrate (e.g., poly-lactide-co-glycolide (PLGA)), fluidizer, antioxidant, citric acid and its salts, and phosphate. Optionally, the pharmaceutical composition could be aerosolized by any suitable approach to facilitate the entry of the pharmaceutical composition into the respiratory tract. For example, the pharmaceutical composition could be administered through a nebulizer or a sprayer, but is not limited thereby.

As for an injection or drip suitable for subcutaneous, intramuscular, peritoneal, or intravenous administration, the pharmaceutical composition could comprise one or more ingredient(s), such as an isotonic solution, a salt-buffered saline (e.g., phosphate-buffered saline or citrate-buffered saline), a hydrotropic agent, an emulsifier, 5% sugar solution, dimethyl acetamide (DMA), PEG100, PEG200, PEG300, PEG400, benzyl benzoate, benzyl alcohol, cremophor, N-Methyl-2-pyrrolidone, and other carriers to provide the pharmaceutical composition as an intravenous infusion, an emulsified intravenous infusion, a powder for injection, a suspension for injection, or a powder suspension for injection, etc. Alternatively, the pharmaceutical composition could be prepared as a pre-injection solid. The pre-injection solid could be provided in a form which is soluble in other solutions or suspensions, or in an emulsifiable form. A desired injection is provided by dissolving the pre-injection solid in other solutions or suspensions or emulsifying it prior to being administered to a subject in need.

As for the form suitable for subcutaneous implantation or interstitial implantation, the pharmaceutical composition provided by the present invention could further comprise one or more ingredient(s), such as an excipient, a stabilizer, a buffer, other carriers, to prepare the pharmaceutical composition in a form of, for example, a wafer, a tablet, a pill, a capsule. Therefore, the pharmaceutical composition could be implanted into a subject.

Depending on the needs, age, body weight, and health conditions of the subject as well as the purpose(s), the composition in accordance with the present invention could be administered at various frequencies, such as once a day, multiple times a day, or once every few days. Depending on the needs of the subject, the composition in accordance with the present invention could be administered at any appropriate dosage. For example, when the composition is administered to a subject by an oral route to treat and/or prevent dementia, the dosage of the composition is 37.5 mg to 50 g per day, and preferably 300 mg to 50 g per day, based on the weight of the second component. However, for patients with more serious dementia, the dosage of the composition could be optionally increased depending on the practical requirements. Moreover, the amounts of the first component and second component in the composition could also be adjusted depending on the practical requirements.

Optionally, the pharmaceutical composition or food composition in accordance with the present invention could further comprise a suitable amount of additives, such as a flavoring agent, a toner, or a coloring agent for enhancing the palatability and the visual perception of the pharmaceutical composition or food composition, and/or a buffer, a conservative, a preservative, an antibacterial agent, or an antifungal agent for improving the stability and storability of the pharmaceutical composition or food composition. In addition, the composition could optionally further comprise one or more other active ingredient(s) to further enhance the effects of the composition, or to increase the application flexibility and application adaptability of preparation thus provided. The other active ingredients should not adversely affect the desired effects of the active ingredients of the present invention (i.e., the first component and the second component).

The food combination in accordance with the present invention could be a health food, a dietary supplement, a functional food, a nutritional supplement food or a special nutritional food. The food combination could be manufactured as dairy products, meat products, breadstuff, pasta, cookies, troche, capsules, fruit juices, tea products, sports beverages, nutrient beverages, etc., but is not limited thereby.

Depending on the age, body weight, and health conditions of the subject, the health food, dietary supplement, functional food, nutritional supplement food and special nutritional food in accordance with the present invention could be taken at various frequencies, such as once a day, multiple times a day, or once every few days. The amount of the first component and second component in the health food, dietary supplement, functional food, nutritional supplement food and special nutritional food in accordance with the present invention could also be adjusted, preferably to the amount that should be taken daily, depending on the specific population. For example, based on the total weight of second component, if the recommended daily dosage for a subject is 500 mg of the second component and each serving of the dietary supplement contains 250 mg of the second component, the subject can take two servings of the dietary supplement per day.

The recommended daily dosage, use standards and use conditions for a specific population (e.g., patients with dementia, patients with cancer, pregnant woman), or the recommendations for a use in combination with another food product or medicament could be labeled on the exterior package of the health food, dietary supplement, functional food, nutritional supplement food and/or special nutritional food in accordance with the present invention. Thus, it is suitable for the users to take the health food, dietary supplement, functional food, nutritional supplement food and/or special nutritional food by him- or herself safely and securely without the instructions of a doctor, pharmacist or related executive.

As mentioned above, the present invention also provides a method for treating and/or preventing dementia, comprising administering to a subject in need thereof an effective amount of the composition described above. The aforementioned “subject in need thereof” refers to a subject suffered from dementia and/or having a high risk of suffering from dementia. In the aforesaid method, the administration type, administration route, administration form, administration frequency, and related uses of the composition are all in line with the above descriptions.

The present invention will be further illustrated in detail with specific examples as follows. However, the following examples are provided only for illustrating the present invention and the scope of the present invention is not limited thereby. The scope of the present invention will be indicated in the appended claims.

### EXAMPLE

### Preparation Example

A. Establishment of Animal Model

Sprague-Dawley (SD) male rats (age: 2-4 months; body weight: 300-500 g) were separated into five groups, and respectively treated by the following conditions:
(1) “Control” group (normal rats; i.e., rats without developing dementia; n=4): the rats were housed in a transparent cage (volume: 43×23×20 cm³; temperature: 25±1° C.; circadian rhythm: 12 hours each of light and darkness, the light was turned on at 6 a.m. and turned off at 6 p.m.; drinking and eating ad libitum) for 8 weeks.
(2) “FAB” group (rats with dementia; n=7): (i) the rats were anaesthetized then fixed on a stereotaxic apparatus; (ii) the skin and muscles covering the rats' skulls were cut open; (iii) an injection needle of Brain infusion kit 2 (purchased from Alzet) was implanted into the lateral ventricle (coordinate: front/back: −1 mm, side: 1 mm, depth: 4.5 mm) by serving the cross suture on the skull as an anchor point based on the rat brain stereotaxic atlas (Paxions and Watson, 1988); (iv) an Osmotic pump (purchased from Alzet), which is filled with FAB solution (comprising 15μmol/L of Aβ₁₋₄₂ protein, 1 mmol/L of ferrous sulfate (FeSO₄) and 12 mmol/L of buthionine sulfoximine (BSO)), was connected to an injection needle and then sutured to the subcutaneous position of the rat's dorsum; and (v) the rats were housed in a transparent cage and continuously perfused with FAB solution for 8 weeks.
(3) “Formulation 1” group (n=3): the rats were subjected to the steps same as those of the “FAB” group, but starting from the 2^{nd} week of step (v), the rats were additionally administered with 500 mg/kg of Formulation 1 composition (containing 10 wt % curcumin, 10 wt % EGCG, 5 wt % apigenin and 75 wt % N-Methyl-2-pyrrolidone solution) via intraperitoneal injection every day.
(4) “Formulation 2” group (n=5): the rats were subjected to the steps same as those of the “FAB” group, but starting from the 2^{nd} week of step (v), the rats were additionally administered with 500 mg/kg of Formulation 2 composition (containing 5 wt % curcumin, 10 wt % EGCG, 5 wt % anthocyanin, 0.1 wt % caffeine and 79.9 wt % N-Methyl-2-pyrrolidone solution) via intraperitoneal injection every day.
(5) “Formulation 3” group (n=4): the rats were subjected to the steps same as those of the “FAB” group, but starting from the 2^{nd} week of step (v), the rats were additionally administered with 500 mg/kg of Formulation 3 composition (containing 5 wt % curcumin, 10 wt % EGCG, 5 wt % anthraquinone, 0.1 wt % caffeine and 79.9 wt % N-Methyl-2-pyrrolidone solution) via intraperitoneal injection every day.

B. Sample Collection

The rats of each group provided by [Preparation Example A] were anaesthetized with 7% chloral hydrate (0.5 ml/100 g-body weight, intraperitoneal injection) and then fixed on a wood board. The rats were subjected to the procedures as below to provide samples for the following experiments:
(a) collecting 3-6 ml blood by cardiac puncture;
(b) cutting the abdominal cavity, diaphragm and thoracic cavity open to expose the heart, inserting the perfusion tube into the left ventricle and then into the aorta ascendens, cutting the right auricle open to release blood, and then replacing the blood with physiological saline;
(c) conducting to a perfusion with 2% paraformaldehyde (dissolving in a 0.1M phosphate buffer (PB); pH 7.3) to fix the tissues;
(d) after completing the fixation, cutting the skull open and collecting the brain tissue; and
(e) placing the brain tissue obtained from step (d) into a culture dish with 0.1M PB buffer, and conducting a coronal slicing by vibratome (TPI, St. Louis, Mo.) to collect the following Tissues (1)-(4):
   Tissue (1): a 1,000 μm thick brain tissue slice obtained by slicing the tissue at the position of 1,000 μm from the injection point (comprising medial septal nucleus);
   Tissue (2): a 1,500 μm thick brain tissue slice obtained by slicing the tissue at the position of 1,500 μm form the frontal hippocampus (comprising hippocampus);
   Tissue (3): two 350 μm brain tissue slices obtained by slicing the tissue at the position of 350 μm from the slicing site of Tissue (2); and
   Tissue (4): the remaining tissues.
[Animal Experiments]

### Example 1

### Influence of the Composition of the Present Invention on the Aβ₁₋₄₂ Protein Aggregation in the Hippocampal CA1 Region

It is known that there are Aβ₁₋₄₂ protein aggregations in the brains of patients with dementia, and those abnormal protein aggregations will impair the neural signal transmission in the brain, thereby, causing damage to the cognitive function and memory function of patients. To investigate the effects of the composition of the present invention on reducing the Aβ₁₋₄₂ protein aggregation, the following experiments were conducted.
(1-1) Treatment of Slices

The Tissues (2) of each group provided by [Preparation Example B] (including “Control” group, “FAB” group, “Formulation 1” group, “Formulation 2” group and “Formulation 3” group) were fixed with 4% trichloroformaldehyde (dissolving in 0.1M phosphate buffered saline) for one day, and then soaked in 30% sucrose solution (dissolving in 0.1M phosphate buffered saline) and serially sliced to a thickness of 30 μm using a freezing microtome.
(1-2) Staining

The slices provided by (1-1) were placed into a 0.1M PBS containing 10% NGS (normal goat serum) and 0.1% Triton and kept for one hour, and then, moved into a PBS containing rabbit anti-Aβ₁₋₄₂ antibody (1:100) and 1% NGS and kept at 4° C. for 20 hours. Thereafter, the slices were washed with PBS thrice, and then, placed into a PBS containing goat anti-rabbit antibody (1:200) and 1% NGS and kept at room temperature for one hour. Finally, the slices were washed with PBS thrice.
(1-3) Pigmentation

The slices provided by (1-2) were placed into a 0.05M Tris buffer (pH 7.4) containing 0.05% DAB (3,3-diaminobenzidine tetrahydrochloride; purchased form Sigma, St. Louis, Mo.) and 0.01% H₂O₂ and kept at room temperature until the color of DAB (i.e., pigment substrate) was revealed. Thereafter, the slices were washed with 0.05M Tris buffer once and then with 0.1M PBS twice.
(1-4) Mounting and Observation

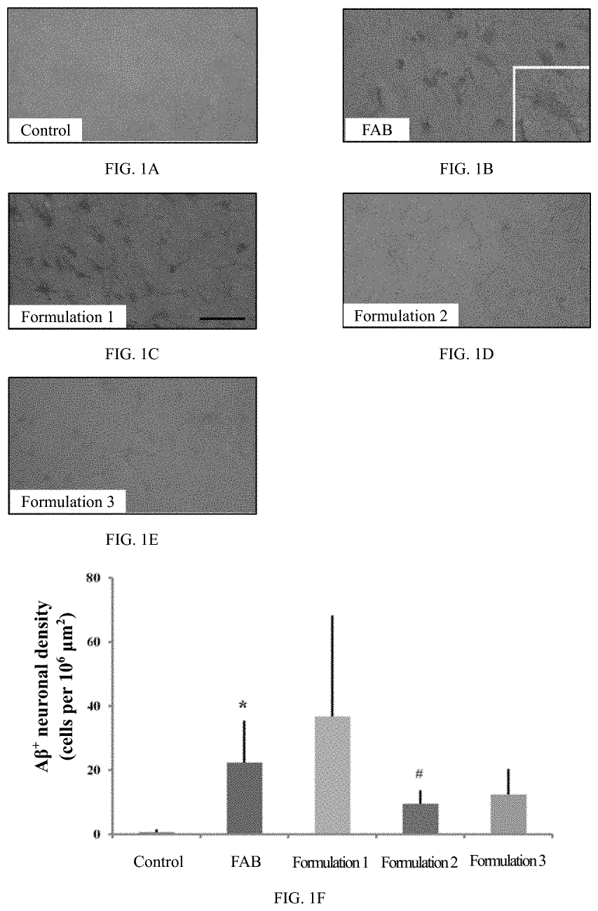

The slices provided by (1-3) were mounted onto the microscope slides and shade dried, then dehydrated by 85%, 95% and 100% (v/v) alcohol sequentially and rendered transparent by xylene. The slides were then coverslipped using Permount. Completed slides were observed by a 10X ocular lens and a 40X objective lens and three photos of the right brain were taken randomly. The results are shown in FIGS. 1A to 1E, wherein FIG. 1F is a bar graph showing the quantified results of FIGS. 1A to 1E.
(1-5) Results Analysis

As shown in FIGS. 1A to 1F, the density of the Aβ₁₋₄₂ protein aggregated in the hippocampal CA1 region of “FAB” group was higher than that of “Control” group. This result indicates that the FAB solution would enhance the aggregation of the Aβ₁₋₄₂ protein, thereby inducing dementia. On the other hand, in comparison with the “FAB” group, the density of the Aβ₁₋₄₂ protein aggregated in the hippocampal CA1 region of “Formulation 1” group did not decrease, but even increase. However, in comparison with the “FAB” group, the density of the Aβ₁₋₄₂ protein aggregated in the hippocampal CA1 region of both the “Formulation 2” group and “Formulation 3” group significantly lower.

The above results indicate that the composition of the present invention (e.g., “Formulation 2” and “Formulation 3”), comprising a first component and a second component, can effectively decrease the density of Aβ₁₋₄₂ protein aggregated in the brain of dementia rat model and help the recovery of neural signal transmission in the brain, and thus, is beneficial to the recovery of cognitive function and memory function of patients with dementia.

### Example 2

### Influence of the Composition of the Present Invention on Microglia Density in Hippocampal CA1 Region

It is known that microglia are a type of macrophage and are responsible for the phagocytosis of pathogens or damaged neurons in the central nervous system. Since there are many damaged neurons and abnormal protein aggregations in the brain of patients with dementia, the microglia will continuously accumulate and remain active, leading to the release of inflammatory activators. However, the excessive inflammatory activators also lead to a neuronal damage, and thus, will exacerbate the symptoms of dementia. To investigate the effects of the composition of the present invention in inhibiting the accumulation and activation of microglia, the following experiments were conducted.
(2-1) Treatment of Slices

The steps described in (1-1) were repeated.
(2-2) Staining

The slices provided by (2-1) were placed into a 0.1M PBS containing 10% NRS (normal rabbit serum) and 0.1% Triton and kept for one hour, and then, moved into a PBS containing goat anti-Ibal antibody (1:1000) and 1% NRS and kept at 4° C. for 18 hours. Thereafter, the slices were washed with 0.1M PBS thrice, and then, placed into a PBS containing rabbit anti-goat antibody (1:200) and 1% NRS and kept at room temperature for one hour. Again, the slices were washed with PBS thrice. Then, the slices were placed into a 0.1M PBS containing Avidin-Biotin-HRP complex (purchased from Vector, Burlingame, Calif.) and kept for one hour.
(2-3) Pigmentation

The slices provided by (2-2) were treated by the same method as described in (1-3).
(2-4) Mounting and Observation

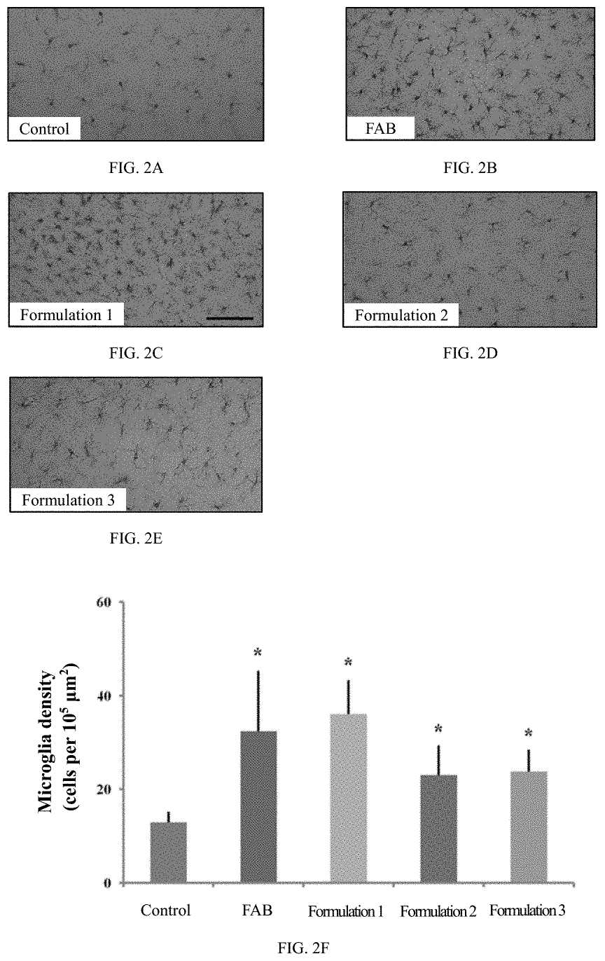

The slices provided by (2-3) were mounted onto the microscope slides and shade dried. Then, they were dehydrated by 85%, 95% and 100% (v/v) alcohol sequentially, and rendered transparent by xylene. The slides were then coverslipped using Permount. Completed slides were observed with a 10X ocular lens and a 20X object lens and three photos of the hippocampal CA1 granular layer in the right brain were taken randomly. The results are shown in FIGS. 2A to 2E, wherein FIG. 2F is a bar graph showing the quantified results of FIGS. 2A to 2E.
(2-5) Results Analysis

As shown in FIGS. 2A to 2F, the microglia density in the hippocampal CA1 region of “FAB” group was higher than that of “Control” group. On the other hand, in comparison with the “FAB” group, the microglia density in the hippocampal CA1 region of “Formulation 1” group did not decrease, but even increase. However, in comparison with “FAB” group, the microglia density in the hippocampal CA1 region of both the “Formulation 2” group and “Formulation 3” group was significantly lower.

The above results indicate that the composition of the present invention (e.g., “Formulation 2” and “Formulation 3”), comprising a first component and a second component, can effectively inhibit the accumulation and activation of microglia in the dementia rat model, and thus, can prevent the neuronal damages and alleviate the symptoms of patients with dementia.

### Example 3

### Influence of the Composition of the Present Invention on Survival Rate of Pyramidal Neurons in Hippocampal CA1 Region

It is known that the hippocampus plays major roles in memory formation and storage; the degeneration or death of pyramidal neurons in the hippocampal CA1 region will cause the occurrence of dementia. In general, it is believed that if the survival rate of pyramidal neurons can be increased, the onset of dementia will be alleviated. To investigate the effects of the composition of the present invention in increasing the survival rate of pyramidal neurons in hippocampal CA1 region, the following experiments were conducted.
(3-1) Treatment of Slices

The Tissues (4) of each group provided by [Preparation Example B] (including “Control” group, “FAB” group, “Formulation 1” group, “Formulation 2” group and “Formulation 3” group) were treated by the same method as described in (1-1).
(3-2) Staining

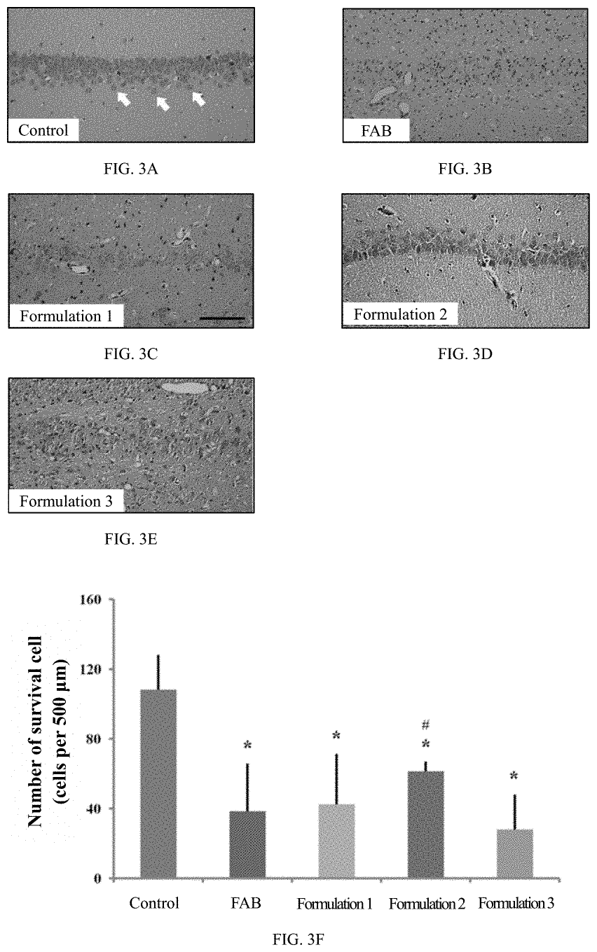

The slices provided by (3-1) were delegated to the Department of Veterinary Medicine, National Chung Hsing University, Taiwan, to perform an H&E stain. The results are shown in FIGS. 3A to 3E, and FIG. 3F is a bar graph showing the quantified results of FIGS. 3A to 3E.
(3-3) Results Analysis

As shown in FIGS. 3A to 3E, the survival rate of pyramidal neurons in the hippocampal CA1 region of the “FAB” group is lower than that of the “Control” group. However, in comparison with the “FAB” group, the survival rate of pyramidal neurons in the hippocampal CA1 region of the “Formulation 2” group was significantly higher. The survival rate of pyramidal neurons in hippocampal CA1 region of the “Formulation 1” group and “Formulation 3” group was not significantly different from that of the “Control” group.

The above results indicate that the composition of the present invention (e.g., “Formulation 2”), comprising a first component and a second component, could effectively increase the survival rate of pyramidal neurons in the dementia rat model, and thus, can delay the onset of dementia.

### Example 4

### Influence of Composition of the Present Invention on Dendritic Spine Density on Distal Apical Dendrite of Pyramidal Neurons in the Hippocampal CA1 region

It is known that a dendrite is a structure responsible for receiving signals. The higher the dendritic spine density, the higher the efficiency of receiving signals. When the dendritic spine density of dendrite is decreased, the cognitive function and memory function would be attenuated. To investigate the effects of the composition of the present invention in increasing the dendritic spine density, the following experiments are conducted.
(4-1) Intracellular dye injection (see such as a literature of Chen et al., “The effect of epidural compression on cerebral cortex: a rat model. J Neurotrauma. 2003 August; 20(8): 767-780.”)

The Tissues (3) of each group provided by [Preparation Example B] (including “Control” group, “FAB” group, “Formulation 1” group, “Formulation 2” group and “Formulation 3” group) were placed into a 0.1M PB buffer containing 10⁻⁷M DAPI (4,6-diamidino-2-phenyl-indole; purchased from Sigma, St. Louis, Mo.) and kept for 30 minutes. Thereafter, the tissues were placed into a chamber with a diameter of 2 cm, covered by a filter paper with a square hole in the center, and then rinsed in a 0.1M PB buffer containing DAPI. Then, the chamber was put under the upright fluorescence microscope (fixed-stage; purchased from Olympus, Tokyo, Japan), and the slices were observed by a 20X ocular lens. The DAPI-stained nuclei with a blue fluorescence could be observed (390-420, FT425, LP450).

A boron-silicate glass tube with an outside/inside diameter of 1.0/0.75 mm was used to produce a glass electrode by using a glass electrode manufacturer (purchased from: Sutter, San Rafael, Calif.; Product no.: P87/PC). The glass electrode (filling length: 1 cm) was filled with LY dye (Lucifer Yellow; purchased from: Sutter, San Rafael, Calif.). Then, the dye-filled electrode was placed onto the silver wire electrode of 3-axial hydraulic micromanipulator (purchased from: Narishige, Tokyo, Japan), and then, the electrode was connected to a microcurrent controller (Axoclamp; purchased from: Axon, Foster City, Calif.).

The following operations were conducted under a fluorescence microscope: (i) the glass electrode was inserted into the fifth layer of somatosensory-motor cortex and the cell bodies in pyramidal neurons in hippocampal CA1 region by using the 3-axial micromanipulator; (ii) the dye was injected into the cells by using a negative microcurrent until the whole dendrite end of cell was filled with dye. To avoid vibrations, the aforesaid apparatus is fixed on an anti-vibration table. The stained tissues were fixed with 4% trichloroformaldehyde (dissolving in 0.1M PB buffer) for more than one day.
(4-2) Treatment of Slices

The tissues provided by (4-1) were sequentially soaked in a solution of 5% sucrose (dissolving in 0.1M PB buffer) (20 minutes), a solution of 20% sucrose and 10% glycerol (dissolving in 0.1M PB buffer) (20 minutes), and a solution of 20% sucrose and 10% glycerol solution (dissolving in 0.1M PB buffer) (20 minutes). After the tissues sank down in the solutions, the tissues were serially sliced to a thickness of 60 μm using a freezing microtome.
(4-3) Staining

The slices provided by (4-2) were placed into a 0.1M PBS containing 2% bovine serum albumin (purchased from: Sigma, St. Louis, Mo.) and kept for one hour. Thereafter, the slices were moved into a PBS containing biotinylated rabbit anti-LY antibody (1:400; purchased from: Molecular Probes, Eugene, Oreg.) and 2% bovine serum albumin and kept at 4° C. for 18 hours. Then, the slices were washed with a 0.1M PBS thrice (10 minutes each time), and put into a 0.1M PBS containing avidin-biotin-horseradish peroxidase complex and kept for one hour. The slices were washed with a 0.1M PBS twice (10 minutes each time) and with 0.05M Tris buffer once (10 minutes each time).
(4-4) Pigmentation

The slices provided by (4-3) were treated by the same method as described in (1-3).
(4-5) Mounting and Observation

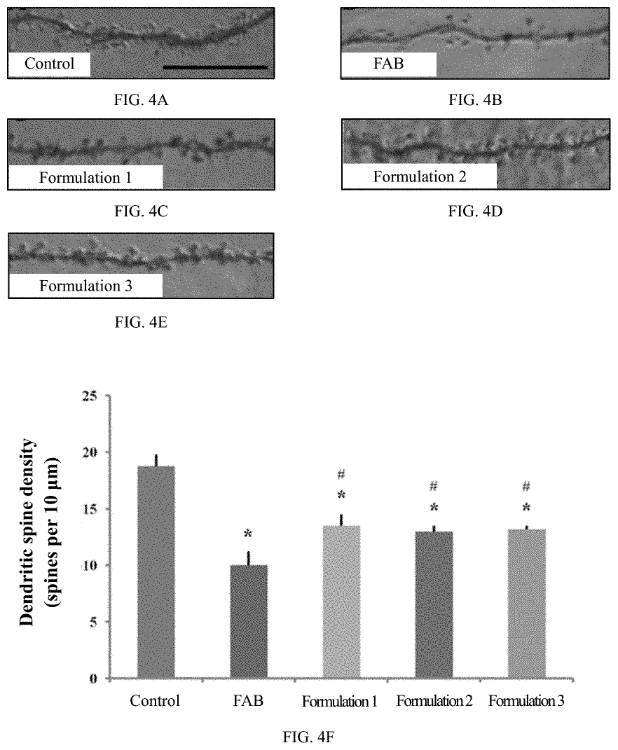

The slices provided by (4-4) were mounted onto the microscope slides and shade dried. Then, they were dehydrated by 85%, 95% and 100% (v/v) alcohol sequentially, and rendered transparent by xylene. The slides were then coverslipped using Permount. Completed slides were observed with a 100X oil immersion lens and the photos of 15 dendrite fragments were taken randomly. Then, the dendritic spine density of distal apical dendrite of pyramidal neurons in the hippocampal CA1 was calculated by setting 10 μm as a unit. The results are shown in FIGS. 4A to 4E, and FIG. 4F is a bar graph showing the quantified results of FIGS. 4A to 4E.
(4-6) Results Analysis

As shown in FIGS. 4A to 4F, the dendritic spine density of distal apical dendrite of pyramidal neurons in the hippocampal CA1 region of the “FAB” group was lower than that of the “Control” group. On the other hand, the dendritic spine density of distal apical dendrite of pyramidal neurons in hippocampal CA1 region of all the “Formulation 1” group, “Formulation 2” group and “Formulation 3” group was higher than that of the “FAB” group.

The above results indicate that the composition of the present invention (e.g., “Formulation 2” and “Formulation 3”), comprising a first component and a second component, could effectively increase the dendritic spine density of distal apical dendrite of pyramidal neurons in the dementia rat model, and thus, is beneficial to the recovery of cognitive function and memory function of patients with dementia.

### Example 5

### Influence of Composition of the Present Invention on Number of Cholinergic Neurons in the Medial Septal Nucleus

It is known that cholinergic neurons secrete the neurotransmitter acetylcholine. The secretion of acetylcholine will decrease along with the decrease in the number of cholinergic neurons. This leads to the degeneration of memory function and the occurrence of dementia. In general, it is believed that if the number of cholinergic neurons in the medial septal nucleus can be increased, the degeneration of memory function will be prevented. To investigate the effects of the composition of the present invention in increasing the number of cholinergic neurons, the following experiments were conducted.
(5-1) Treatment of Slices

The Tissues (1) of each group provided by [Preparation Example B] (including “Control” group, “FAB” group, “Formulation 1” group, “Formulation 2” group and “Formulation 3” group) were treated by the same method as described in (1-1).
(5-2) Staining

The slices provided by (5-1) were placed into a 0.1M PBS containing 10% NHS (normal horse serum) and 0.1% Triton and kept for one hour. Then, they were moved into a PBS containing goat anti-ChAT antibody (1:100) and 1% NHS and kept at 4° C. for 18 hours. Thereafter, the slices were washed with 0.1M PBS thrice. Then, they were placed into a PBS containing rabbit anti-goat antibody (1:200) and 1% NHS and kept at room temperature for one hour. Finally, the slices were washed with PBS thrice.
(5-3) Pigmentation

The slices provided by (5-2) were treated by the same method as described in (1-3).
(5-4) Mounting and Observation

The slices provided by (5-3) were mounted onto microscope slides and shade dried. Then, they were dehydrated by 85%, 95% and 100% (v/v) alcohol sequentially, and rendered transparent by xylene. The slides were then coverslipped using Permount. Completed slides were observed with a 10X ocular lens and 10X object lens, and three photos of the right brain were taken randomly.

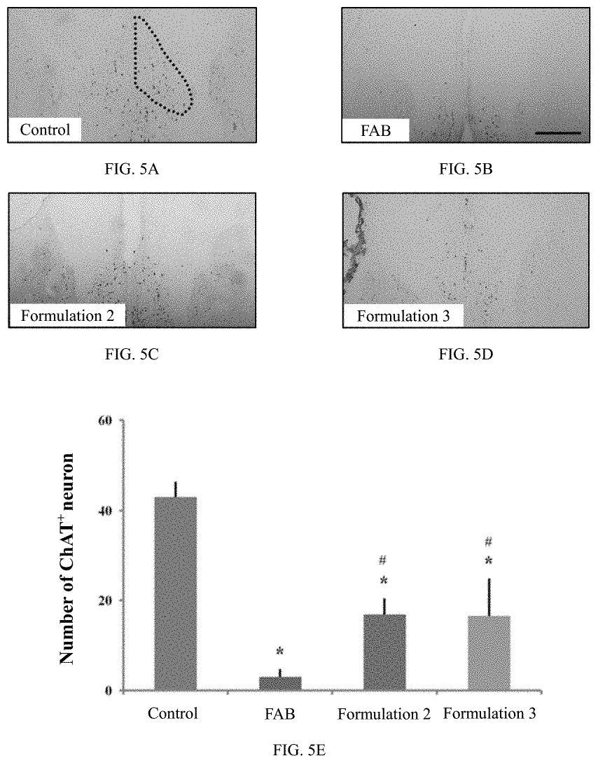

The results are shown in FIGS. 5A to 5D. FIG. 5E is a bar graph showing the quantified results of FIGS. 5A to 5D.
(5-5) Result Analysis

As shown in FIGS. 5A to 5E, the number of cholinergic neurons in the medial septal nucleus of the “FAB” group was significantly lower than that of the “Control” group. However, in comparison with the “FAB” group, the number of cholinergic neurons in the medial septal nucleus of both the “Formulation 2” group and “Formulation 3” group was significantly higher.

The above results indicate that the composition of the present invention (e.g., “Formulation 2” and “Formulation 3”), comprising the first component and second component, could effectively increase the number of cholinergic neurons in the medial septal nucleus of the dementia rat model, and thus, is beneficial to the recovery of cognitive function and memory function of patients with dementia.
[Human Experiments]

### Example 6

### Influence of Composition of the Present Invention for Patients with Dementia

(6-1) Patient #1

Table 1 is a Neuropsychiatric Inventory Questionnaire (NPI-Q), obtained from the evaluator by evaluating the behaviors and mental state of patients (totally 12 items, scoring according to the severity and occurrence frequency). The score of NPI-Q could be a classification standard as the degree of dementia, wherein a score less than 10 belongs to “normal”, a score between 10 and 40 belongs to “mild to moderate dementia”, and a score more than 40 belongs to “moderate to severe dementia.”

The patients with Alzheimer's disease took 2 capsules (each contains 400 mg of the composition of the present invention; ratio of the amount of the components: curcumin=24.9 wt %, EGCG=49.7 wt %, anthocyanin=24.9 wt %, caffeine=0.5 wt %) every day for three months. The NPI-Q scores of patients were recorded at the beginning of experiment (i.e., before taking drugs), 1^{st} month after the experiment began (i.e., consistently taking drugs for one month), and the end of experiment (i.e., consistently taking drugs for three months). The results are shown in Table 1 as below.

TABLE 1

| | before | taking drugs | taking drugs for |
|---|---|---|---|
| | taking drugs | for one month | three months |
| Delusions | 2 | 0 | 0 |
| Hallucinations | 0 | 0 | 0 |
| Agitation/Aggression | 0 | 0 | 0 |
| Depression/Dysphoria | 4 | 0 | 1 |
| Anxiety | 0 | 0 | 0 |
| Elation/Euphoria | 0 | 0 | 0 |
| Apathy/Indifference | 12 | 12 | 0 |
| Disinhibition | 0 | 0 | 0 |
| Irritability/Lability | 0 | 0 | 0 |
| Motor Disturbance | 0 | 0 | 0 |
| Nighttime Behaviors | 12 | 12 | 8 |
| Appetite/Eating | 12 | 12 | 0 |
| Total score | 42 | 36 | 9 |

As shown in Table 1, in comparison with the state before taking drugs, the behavior and mental state of patient were significantly improved after taking the composition of the present invention for one month. The NPI-Q score of patients was decreased to less than 10 (i.e., considering as “normal”) after taking the composition of the present invention for three months. The previous results indicate that the composition of the present invention, comprising the first component and second component, could be used for treating and/or preventing dementia.
(6-2) Patient #2

The patients with Alzheimer's disease took 2 capsules (each contains 400 mg the composition of the present invention ratio of the amount of the components: curcumin=24.9 wt %, EGCG=49.7 wt %, anthocyanin=24.9 wt %, caffeine=0.5 wt %) every day for five months. At the beginning of the experiment (i.e., before taking drugs) and the end of the experiment (i.e., consistently taking drugs for five months), the scores of the patients in the following evaluations were recorded: Mini-Mental State Examination (MMSE), Short Portable Mental State Questionnaire (SPMSQ), Behavior Test, Geriatric Depression Scale (GDS), Barthel index, and Instrumental Activities of Daily Living (IADL). The results are shown in Table 2 as below.

TABLE 2

| Questionnaire | | | Behavior | | Barthel | |
|---|---|---|---|---|---|---|
| Score | MMSE | SPMSQ | Test | GDS | index | IADL |
| Before taking | 18 | 3 | 0 | 4 | 100 | 82.5% |
| drugs | | | | | | |
| Taking | 18 | 2 | 0 | 4 | 100 | 77.5% |
| drugs for | | | | | | |
| five months | | | | | | |

The evaluation items of SPMSQ include orientation, personal information, remote memory, arithmetic ability, etc (10 items in total; the score was increased 1 point for each wrong answer). IADL is used to evaluate daily matters which are more complex and need a higher ability to do so (including shopping, clothes washing, finance handling, etc). Every item of IADL could be divided into 3 to 5 degrees, but the score is only calculated as “1” or “0”. For example, one which can wash all personal clothes and the other which only can wash small clothes are divided into different degrees. Both of them are deemed as having the ability to wash clothes, and so, the score of them increased by 1 point. To reflect the state of the patient via the final IADL score precisely, the IADL percentage of a subject is evaluated by setting the score of a patient without any abilities of independently living (i.e., every daily matter is classified as the highest degree) as 100%. The lower the IADL percentage, the higher the abilities of independently living.

As known in Table 2, after taking the composition of the present invention for five months, both the SPMSQ score and IADL percentage of the subject significantly decreased. The previous results indicate that after the patients taking the composition of the present invention for five months, their memory and the ability of independently living significantly improved.
[Adduct Synthesis]

### Example 7

### The First Component and Second Component were Combined as an Adduct

(7-1) First Component: Caffeine/Second Component: Rosemaric Acid

0.1 mmol of rosemaric acid (dissolving in toluene) was mixed with 0.11 mmol caffeine at room temperature, and the mixture thus obtained was put into a 10 ml round bottom flask that contained 2 to 3 ml toluene. Then, the flask was set up in a reflux apparatus and heated to 60° C. for one hour to conduct an addition reaction (during the one-hour period, a little portion of mixture was taken from the flask regularly and examined by a thin layer chromatography) and provide a Caffeinium rosemarinate adduct in which caffeine and rosemaric acid are combined through a hydrogen bond. The addition reaction is shown as follows:

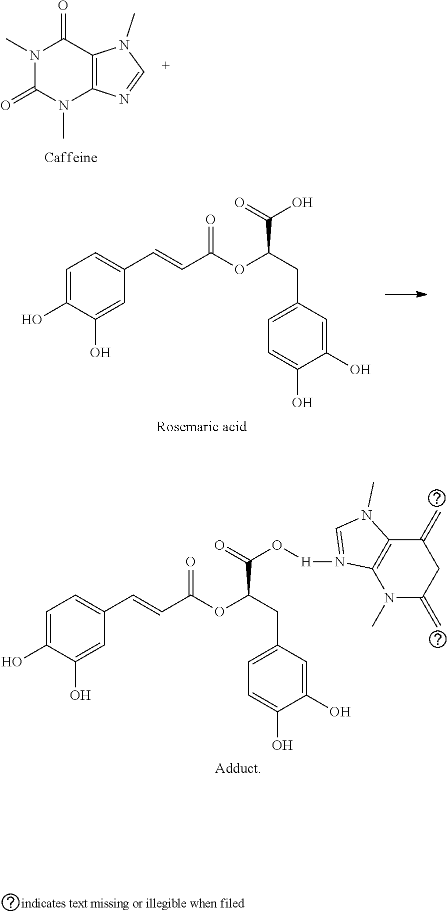

After the reaction was completed and the mixture was cooled down to room temperature, the mixture was divided into two portions, i.e., the first portion and the second portion that were separately subjected to the following two procedures to isolate the Caffeinium rosemarinate adduct. The first portion was lyophilized to remove the solvent, the solid thus obtained was mixed with light petroleum ether to provide a mixture, the mixture was shaken to remove the excess amount of caffeine and then was subject to a centrifugation, and the solid thus obtained was dried at 50° C. until constant weight. The second portion was diluted with light petroleum ether and the material settled on the bottom was collected. The collected material was mixed with light petroleum ether to provide a mixture and the mixture was shaken to remove the excess amount of caffeine and then was subject to a centrifugation, and the solid thus obtained was dried at 50° C. until constant weight.
(7-2) First Component: Caffeine/Second Component: Kaempferol

0.1 mmol of kaempferol (dissolving in toluene) was mixed with 0.11 mmol caffeine at room temperature. The mixture thus obtained was put into a 10 ml round bottom flask that contained 2 to 3 ml toluene. Then, the flask was set up in a reflux apparatus and heated to 60° C. for one hour to conduct an addition reaction (during the one-hour period, a little portion of mixture was taken from the flask regularly and examined by a thin layer chromatography) and provide a kaempferol-caffeine adduct in which caffeine and kaempferol are combined through a hydrogen bond. The addition reaction is shown as follows:

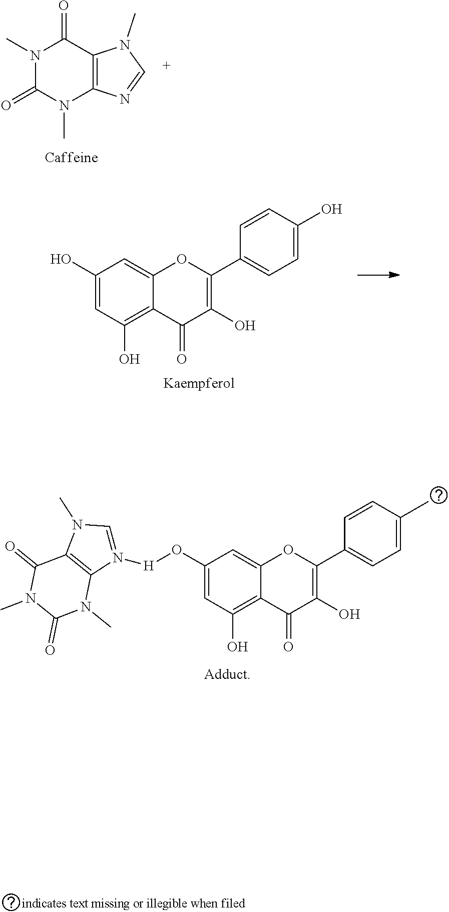

After the reaction was completed and the mixture was cooled down to room temperature, the mixture was subjected to the following procedure to isolate the adduct. After removing almost all of the solvent via lyophilization, the remaining mixture was purified by a preparative thin-layer chromatography to remove the excessive caffeine. The band was scraped from the initial point and washed with toluene. Finally, the mixture of the band and toluene was filtered and centrifuged, and then dried at 50° C. until constant weight.
(7-3) First Component: Caffeine/Second Component: Quercetin

Quercetin (101.46 mg, 0.3 mmol), caffeine (58.23 mg, 0.3 mmol) and 2 ml methanol were mixed under stirring for 12 hours at a nitrogen atmosphere and room temperature to conduct an addition reaction, and the mixture thus obtained was filtered to provide a solid. Then, the solid was dried at 150° C. under vacuum for 3 hours, thereby, providing a quercetin-caffeine adduct “QUECAF” (melting point: 245.95° C.) in which caffeine and quercetin are combined through a hydrogen bond. The addition reaction is shown as follows:

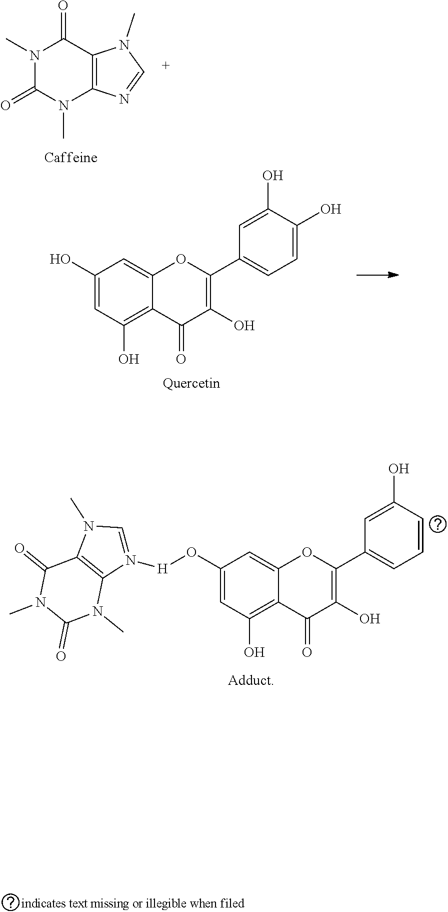

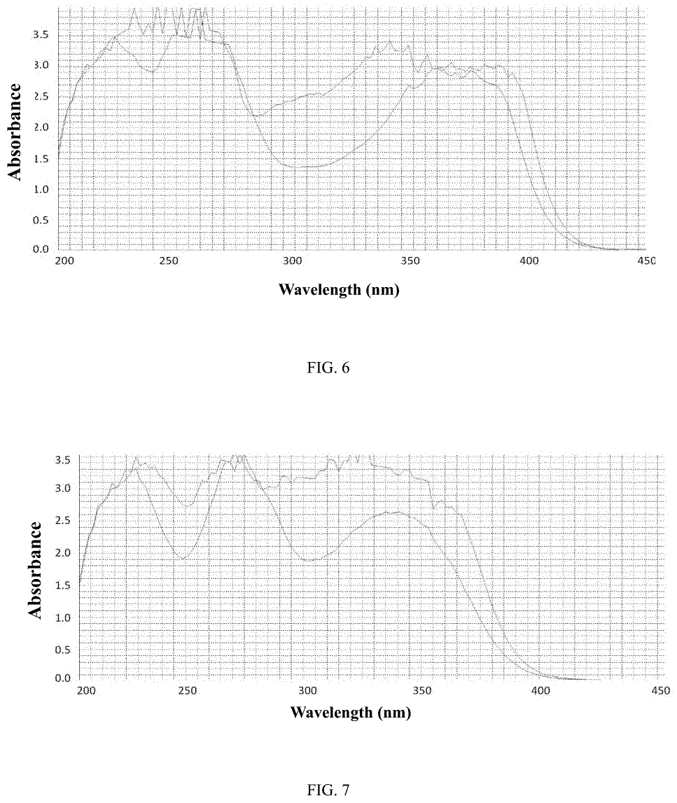

The quercetin and the above quercetin-caffeine adduct were tested by Gold S53 UV-VIS spectrometer to obtain their UV-VIS spectrum. The results are shown in FIG. 6. As shown in FIG. 6, in comparison with the quercetin (red line), the absorbances of the quercetin-caffeine adduct (blue line) at a wavelength of 250 nm and 300 nm are both lower.
(7-4) First Component: Caffeine/Second Component: Delphinidin Chloride

Delphinidin chloride (66.3 mg, 0.1 mmol), caffeine (19.4 mg, 0.1 mmol) and 1 ml water were mixed under stirring for 16 hours at a nitrogen atmosphere and room temperature to conduct an addition reaction. The mixture thus obtained was lyophilized to remove the solvent and provide a “delphinidin chloride-caffeine adduct” (storing in dark) in which caffeine and Delphinidin chloride are combined through a hydrogen bond. The addition reaction is shown as follows:

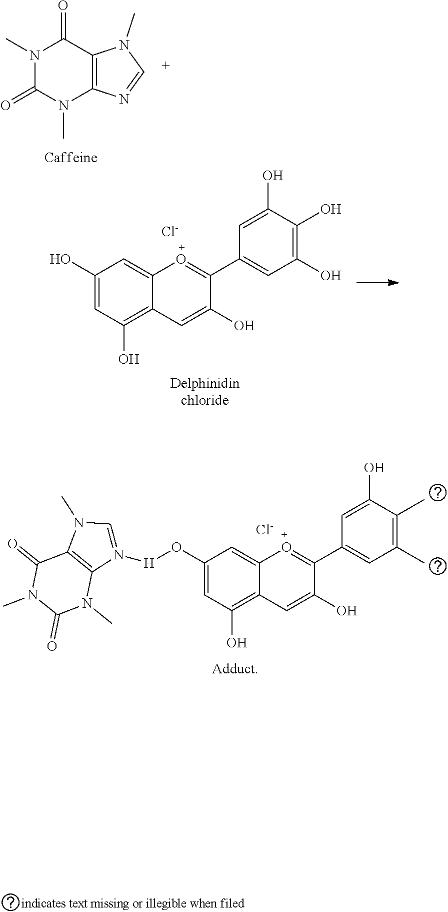

(7-5) First Component: Caffeine/Second Component: Apigenin

Apigenin (81.07 mg, 0.3 mmol), caffeine (58.23 mg, 0.3 mmol) and 2 ml methanol were mixed under stirring for 12 hours Under a nitrogen atmosphere and room temperature to conduct an addition reaction, and the mixture thus obtained was filtered to provide a solid. Then, the solid was dried at 150° C. under vacuum for 3 hours, thereby providing a “apigenin-caffeine adduct” in which caffeine and apigenin are combined through a hydrogen bond. The addition reaction is shown as follows:

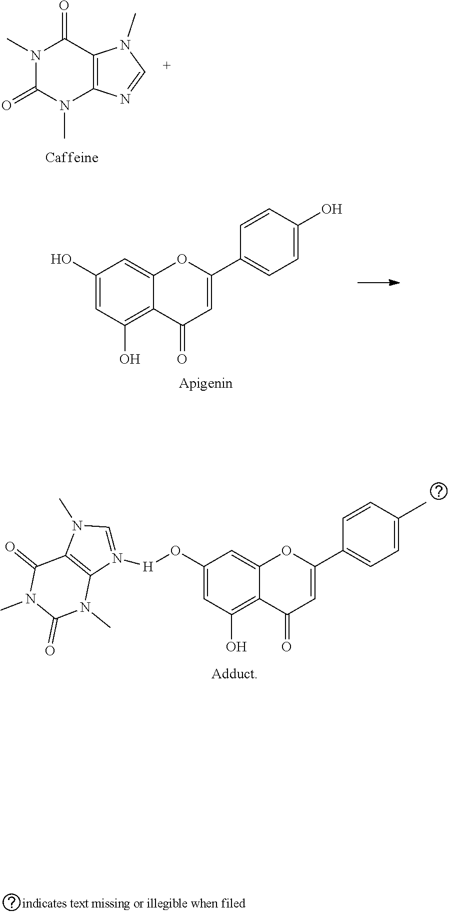

The apigenin and the above apigenin-caffeine adduct were tested by Gold S53 UV-VIS spectrometer to obtain their UV-VIS spectrum. The results are shown in FIG. 7. As shown in FIG. 7, in comparison with the apigenin (red line), the absorbances of apigenin-caffeine adduct (blue line) at a wavelength of 250 nm and 300 nm are both lower.

Unless otherwise indicated herein, the percentage (%) recited in the above examples refers to a ratio of weight/volume (w/v). For example, a 30% sucrose solution refers to that a 100 ml solution (comprising solute and solvent) contains 30g sucrose.

The results of above animal experiment show that the composition of the present invention, comprising a first component and a second component (wherein the first component and the second component can optionally combined to form an adduct), is effective in reducing the density of Aβ₁₋₄₂ protein aggregation, inhibiting the accumulation and activation of microglia, increasing the survival rate of pyramidal neuron, increasing the dendritic spine density of distal apical dendrite of pyramidal neurons, and increasing the number of cholinergic neurons in the medial septal nucleus. Thus, the composition of the present invention can prevent the neuronal damage, assist in recovering the neurotransmission in the brain, prevent the degeneration of memory function in patients with dementia, assist in recovering cognitive and memory function, alleviate the symptoms of patient with dementia, and delay the onset of dementia. Furthermore, the results of human experiment show that the composition of the present invention, comprising a first component and a second component, is effective in improving the behaviors and mind states of patients with dementia, as well as is effective in improving the abilities of memory and independent living in patients with dementia. Therefore, the composition of the present invention can be used to treat and/or prevent dementia.

What is claimed is:

1. A composition, comprising a first component and a second component, wherein the first component is at least one of xanthine and a derivative of xanthine, and the second component is at least one of diarylheptanoid, a derivative of diarylheptanoid, catechin, an ester of catechin, flavonoid, and isoflavonoid, and wherein the first component is optionally combined with the second component as an adduct.

2. The composition as claimed in claim 1, wherein the first component is at least one of caffeine, aminophylline, 3-isobutyl-1-methylxanthine (IBMX), paraxanthine, pentoxifylline, theobromine, and theophylline.

3. The composition as claimed in claim 1, wherein the diarylheptanoid is curcumin.

4. The composition as claimed in claim 1, wherein the derivative of diarylheptanoid is at least one of curcuminoid, demethoxycurcumin, and bisdemethoxycurcumin.

5. The composition as claimed in claim 1, wherein the ester of catechin is epigallocatechin gallate (EGCG).

6. The composition as claimed in claim 1, wherein the flavonoid is at least one of apigenin, anthocyanin, anthraquinone, quercetin, and luteolin.

7. The composition as claimed in claim 1, wherein the isoflavonoid is at least one of genistein, daidzein, and glycitein.

8. The composition as claimed in claim 1, further comprising a third component, wherein the third component is at least one of carotenoid, zeaxanthin, lycopene, carotene, carotenoid crocetin, carotenoid acid, and ω-3 fatty acid.

9. The composition as claimed in claim 1, wherein based on the total weight of the first component and the second component, the amount of the first component ranges from 0.025 wt % to 25 wt %.

10. A method for treating and/or preventing dementia, comprising administering an effective of the composition as claimed in claim 1 to a subject in need thereof.

11. The method as claimed in claim 10, wherein the first component is at least one of caffeine, aminophylline, 3-isobutyl-1-methylxanthine (IBMX), paraxanthine, pentoxifylline, theobromine, and theophylline.

12. The method as claimed in claim 10, wherein the diarylheptanoid is curcumin.

13. The method as claimed in claim 10, wherein the derivative of diarylheptanoid is at least one of curcuminoid, demethoxycurcumin, and bisdemethoxycurcumin.

14. The method as claimed in claim 10, wherein the ester of catechin is epigallocatechin gallate (EGCG).

15. The method as claimed in claim 10, wherein the flavonoid is at least one of apigenin, anthocyanin, anthraquinone, quercetin, and luteolin.

16. The method as claimed in claim 10, wherein the isoflavonoid is at least one of genistein, daidzein, and glycitein.

17. The method as claimed in claim 10, wherein the composition further comprises a third component and the third component is at least one of carotenoid, zeaxanthin, lycopene, carotene, carotenoid crocetin, carotenoid acid, and ω-3 fatty acid.

18. The method as claimed in claim 10, wherein based on the total weight of the first component and the second component, the amount of the first component ranges from 0.025 wt % to 25 wt %.

19. The method as claimed in claim 10, wherein the dementia is at least one of Alzheimer's disease, and Parkinson's disease.
